(19) United States
(12) Patent Application Publication
(10) Pub. No.: US 2006/0286289 A1
(43) Pub. Date: Dec. 21, 2006

(54) Method of intraoperative coating therapeutic agents onto sutures

(76) Inventors: Rita Prajapati, Quincy, MA (US); Steven Bowman, Sherborn, MA (US); Mark Timmer, Jersey City, NJ (US); Patrick De Deyne, Duxbury, MA (US); Joseph Hammer, Hillsborough, NJ (US); Chunlin Yang, Belle Mead, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/153,285

(22) Filed: Jun. 15, 2005

(51) Int. Cl. A61F 13/02

(52) U.S. Cl. 427/2.31

(57) ABSTRACT

Intra-operative coating of sutures with therapeutic proteins, particularly growth factors such as rhGDF-5. including contacting a suture to a device containing a therapeutic agent.

### BACKGROUND OF THE INVENTION

The journal and patent literature describe the use of rhGDF-5 for its ability to form tendon, cartilage, bone and ligament-like structures. For example, Rickert et al., Growth Factors, 19, 2001, 115-126, discloses the use of rHGDF-5 upon sutures to stimulate tendon healing in an Achilles tendon model in rats. However, the method of coating the GDF-5 to the sutures is described as simple pipetting of a solution containing GDF-5 onto the suture. In fact, the literature in general does not disclose methods of coating growth factors onto sutures. See for example, U.S. Pat. No. 5,658,882 (Celeste); U.S. Pat. No. 6,187,742 (Wozney); U.S. Pat. No. 6,284,872 (Celeste II); U.S. Pat. No. 6,719,968 (Celeste III); and US Published Patent Application No. 2004/0146923 (Celeste IV).

Coated sutures and implants (with collagen, butyric acid and a variety of growth factors) have been used in soft tissue repair. See, for example, Mazzocca, AAOS Abstract #338, 2005; Wright, 50^{th} ORS, #1234, 2004; Petersen, 51^{st} ORS, #0076, 2005; Schmidmaier, J. Biomedical Materials Res (Appl Biomat) 58, 449-455, 2001. These papers report promising in vitro and in vivo data. However, implantation of these implants into humans using these techniques are not currently possible, as in vitro models require further development and additional data are required to better characterize the in vivo models.

Wright, supra, reports the use of butyric acid treated silicon coated sutures (Tricon—India) in bilateral meniscal tears in an in vivo sheep model. Wright reports that tears repaired with coated sutures possessed new and repaired tissue including neo-angiogenesis at the repair site. This study demonstrates the potential for connective tissue repair. However, as the study reported on by Wright lasted only 6 weeks, long-term follow-up data are lacking.

Petersen, supra, reports the use of an in vivo sheep model, wherein local application of VEGF using poly(D,L lactide)-sutures stimulated proliferation of blood vessels but did not show enhanced meniscus healing.

Sutures coated with antimicrobials are commercially available for clinical use. At present, polyglactin sutures coated with antibiotics sold under the tradename Coated VICRYL PLUS (polyglactin 910) Suture (Ethicon, Somerville, N.J.) is the first and only antibacterial suture approved by the FDA for inhibiting the colonization of bacteria, which causes the majority of surgical site infections. VICRYL PLUS suture creates an inhibitory zone around the suture in which bacteria are prevented from making colonies. See Rothenberger, Surgical Infection Society Journal (Supp) December 2002, pp. 579-87, and Mangram, Infection control and Hospital Epidemiology, 1999, 20(40, 247-280. VICRYL PLUS suture contains a bacteriostat sold under the tradename IRGACARE MP* (Ciba Specialty Chemicals Corp., Tarrytown, N.Y.), the most pure form of triclosan, a proven, broad spectrum antibacterial drug used effectively in consumer products for more than 30 years. VICRYL PLUS suture is indicated for use in general soft tissue approximation and/or ligation, except for ophthalmic, cardiovascular and neurological tissues.

It is an object of the present invention to allow for the intra-operative coating of sutures with therapeutic agents such as growth factors and/or antibiotics.

### SUMMARY OF THE INVENTION

The present inventors have developed a number of devices and procedures for the intra-operative coating of sutures with therapeutic agents such as growth factors.

Therefore, in accordance with the present invention, there is provided a method of coating a suture, comprising the steps of:
a) providing a device containing a therapeutic agent, and
b) contacting the suture to the device.

In a first embodiment of the present invention, a therapeutic agent (such as a lyophilized growth factor) is contained within a vessel having a porous body (such as a sponge) at one end and a fluid (such as saline) is introduced into the vessel to form a solution comprising the therapeutic agent that also wets the sponge. The wetted porous body may then be contacted with a suture to coat the suture with the solution.

Therefore, in accordance with the present invention, there is provided a device for coating a suture, comprising:
a) a vessel having an open end,
b) a porous body enclosing the open end of the vessel; and
c) a therapeutic protein contained within the vessel.

Also in accordance with the present invention, there is provided method of coating a suture, comprising the steps of:
a) providing the device of the first embodiment containing a therapeutic agent,
b) injecting a fluid into the vessel to produce a solution comprising reconstituted solution,
c) contacting the solution with the porous body to wet the porous body,
d) contacting the suture with the wetted porous body.

In a second embodiment of the present invention, an applicator has a porous body (such as a sponge) at one end while the fluid (such as a saline solution) and the therapeutic agent (such as a lyophilized growth factor) are separated within the vessel by a breakable seal. To use the device, the seal is broken thereby allowing to reconstitute the therapeutic agent into a solution, which subsequently wets the porous body. The wet porous body may then be used to coat a suture or other implant.

Therefore, in accordance with the present invention, there is provided a device for coating a suture, comprising:
a) a vessel having an open end and an inner wall,
b) a porous body enclosing the open end of the vessel;
c) a breakable seal disposed upon the inner wall of the vessel defining a first chamber and a second chamber,
d) a fluid contained within the first chamber, and
e) a therapeutic protein contained within the second chamber.

Also in accordance with the present invention, there is provided a method of coating a suture, comprising the steps of:
a) providing the device of the second embodiment containing a therapeutic agent,
b) breaking the seal between the chamber to mix the fluid and the therapeutic agent and wet the porous body, and.
c) contacting the suture with the wetted porous body.

In a third embodiment of the present invention, an applicator has a roller ball at one end and a chamber containing a fluid comprising the therapeutic agent (such as a solution comprising a growth factor). A suture is passed along the periphery of the roller ball that contacts the solution, thereby coating the suture.

Therefore, in accordance with the present invention, there is provided a device for coating a suture, comprising:
a) a vessel having an open end having an opening, a closed end, and an inner wall defining a chamber,
b) a roller ball disposed within the opening;
c) an entry recess and an exit recess extending into the inner wall at the opening,
d) a fluid comprising a therapeutic agent contained within the chamber, and
e) a suture having i) a first end portion extending out of the entry recess, ii) an intermediate portion passing through the vessel and contacting the solution and iii) a second end portion exiting the vessel through the exit recess.

Also in accordance with the present invention, there is provided a method of coating a suture, comprising the steps of:
a) providing the device of the third embodiment, and
b) passing the intermediate portion of the suture through the exit recess.

In a fourth embodiment, a fluid (such as a thermoreversible gel) loaded with growth factor is contained within a vessel having two opposing openings in its opposing end portions. A suture is passed through the fluid via each opening, thereby coating the suture. When the fluid is a thermoreversible gel, it coats the suture in its liquid state and then solidifies on the suture in its gel state.

Therefore, in accordance with the present invention, there is provided a device for coating a suture, comprising:
a) a vessel having a first open end and a second open end, and a chamber therebetween,
b) a fluid comprising a therapeutic agent contained within the chamber of the vessel, and
c) a suture having i) a first end portion extending out of the first open end, ii) an intermediate portion passing through the chamber and contacting the fluid and iii) a second end portion exiting the vessel through the second open end.

Also in accordance with the present invention, there is provided a method of coating a suture, comprising the steps of:
a) providing the device of the fourth embodiment,
b) heating the thermoreversible gel above its gelling point, thereby coating the intermediate portion of the suture, and
c) pulling the second end portion of the suture in order to pass the intermediate portion of the suture coated with the solution through the second open end.

In a fifth embodiment, there is provided a squeezable container having an opening. When the device is squeezed, the hterapeutic agent is forced out of the opening and onto the suture.

Therefore, in accordance with the present invention, there is provided device for coating a suture, comprising:
a) a vessel having a flexible wall defining a chamber, an end surface, and a first opening in the end surface in fluid communication with the chamber, and
b) a fluid containing a therapeutic protein contained within the chamber of the vessel.

The present invention offers a novel method for creating coated sutures intra-operatively. In addition, it provides surgeons with the flexibility to (i) determine the desired amount of therapeutic agent to administer, and (ii) to select the length of suture that requires coating. Moreover, the present invention will provide many advantages over the conventional methods of coating sutures:

First, it will allow for sterile, intra-operative delivery of therapeutic agents (such as growth factors like rhGDF-5) onto sutures for immediate surgical use.

Second, the present invention will provide the surgeon with an ability to vary the dose of the therapeutic agent present on the suture.

Third, the present invention will provide the surgeon with the ability to coat sutures of different sizes and needles types. This allows the surgeon to coat portions of a single suture with the therapeutic agent (such as a growth factor), more specifically in areas where its contact with the host tissue is intended to have a therapeutic effect and leave other portions of the same suture uncoated.

Fourth, the present invention will provide the surgeon with the potential for providing sterile long-term storage of vials of therapeutic agents (such as growth factors like rhGDF-5) at either frozen, refrigerated or at room temperature.

Fifth, the present invention will provide the surgeon with the potential for providing growth factor-coated sutures for soft tissue repair, such as but not limited to repair of anterior cruciate ligament (ACL), medial collateral ligament of the knee or elbow, reconstruction of the anterior shoulder capsule to enhance anterior stability, reattachment of tendons especially near the bony insertion, reconstruction of the lateral joint capsule of the ankle, meniscus, Achilles tendon and rotator cuff tears.

Sixth, the present invention will provide the surgeon with the potential for providing new ways of using therapeutic agents such as growth factors independent of a specific implant.

### DESCRIPTION OF THE FIGURES

FIGS. 1a-1d are cross-sections of the first embodiment of the present invention, whereby a fluid is introduced in a vessel containing a therapeutic agent and the resulting solution is applied through a porous body.

FIGS. 2a-2d are cross-sections of the second embodiment of the present invention, whereby a fluid and a therapeutic agent are separated in the same vessel by a breakable seal.

FIGS. 3a and 3b are cross-sections of the third embodiment of the present invention, whereby a roller ball assists in the movement of a suture through a vessel containing a solution comprising a therapeutic agent.

FIG. 4 is a cross-section of the fourth embodiment of the present invention, whereby a suture is pulled through a vessel containing a fluid containing a therapeutic agent.

FIG. 5 is a cross-section of a fifth embodiment of the present invention, wherein a vessel having a flexible wall is squeezed to push a fluid containing a therapeutic agent through an opening in the vessel.

FIG. 6 describes a woven composite suture comprising a first suture comprising collagen and a second suture.

FIG. 7 describes a composite mesh comprising a first plurality of suture comprising collagen and a second plurality of sutures.

### DETAILED DESCRIPTION OF THE INVENTION

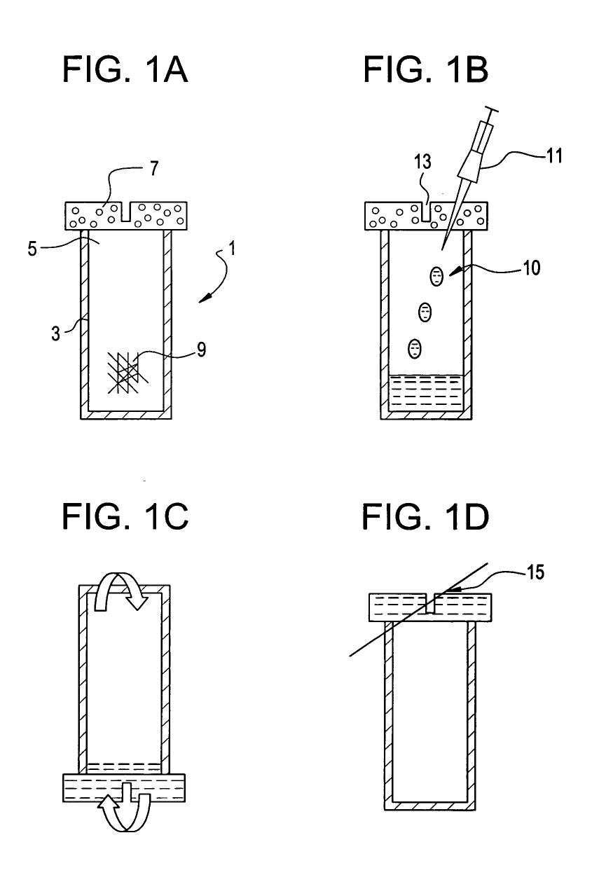

Now referring to FIGS. 1a-1d, there is provided a device 1 for coating a suture, comprising:
a) a vessel 3 having an open end 5,
b) a porous body 7 (such as a sponge) enclosing the open end of the vessel; and
c) a therapeutic agent (such as a growth factor 9 contained within the vessel.

In preferred first embodiments, a sterile vial contains a lyophilized growth factor such as rhGDF-5. Prior to use, the vial is stored at frozen, refrigerated or at room temperature so as to avoid denaturing the protein. The lyophilized rhGDF-5 is reconstituted with sterile saline 10, that has been introduced through the sponge, for example, through a syringe 11 to the desired concentration. Next, the vial is gently mixed. Then, the vial is inverted to allow the solution to soak into the sterile sponge that is present at the open end of the vial. In preferred embodiments, the sterile sponge is the type used in a DERMABOND™ Topical Skin Adhesive applicator (Closure Medical Corp., Raleigh, N.C.). A sterile suture is then placed or rubbed over the saturated sponge to coat the suture with rhGDF-5. In this embodiment, the sponge has a recess 13 through which the suture 15 is passed to allow for peripheral coating of the suture. The sutures are then air-dried to remove the water from the saline solution, thereby leaving rhGDF-5 adhered to the suture. The coated suture can then be used immediately for surgery.

In other preferred first embodiments, a thermoreversible gel is used as the fluid.

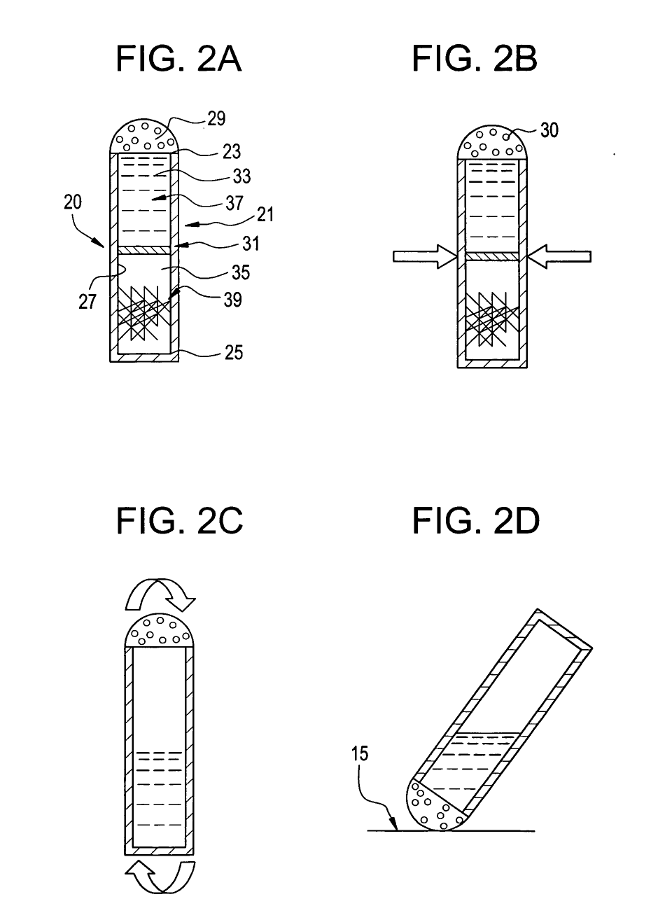

Now referring to FIGS. 2a-2d, there is provided a device 20 for coating a suture, comprising:
a) a vessel 21 having an open end 23, a closed end 25 and an inner wall 27,
b) a porous body 29 (such as a sponge) enclosing the open end of the vessel;
c) a breakable seal 31 disposed upon the inner wall of the vessel defining a first chamber 33 and a second chamber 35,
d) a fluid 37 contained within the first chamber, and
e) a therapeutic agent 39 contained within the second chamber.

In preferred second embodiments, lyophilized rhGDF-5 is the therapeutic agent that is placed in a sterile, flexible vial having a breakable seal that defines two chambers. The rhGDF-5 is stored in one chamber and the second chamber contains a predetermined volume of a fluid such as sterile saline or a thermoreversible gel. Intra-operatively, the flexible vial is squeezed at the location of the breakable seal, thereby breaking the seal between the two chambers and allowing communication between the two chambers and allowing the materials in the chambers to mix. The vial is gently inverted to allow the rhGDF-5 containing solution to soak into the porous body (preferably a domed sponge similar to that of a DERMABOND® applicator). The rhGDF-5 solution is released onto a suture 15 by placing pressure on the domed sponge. Preferably, the rhGDF-5 solution is released onto the sutures by applying constant pressure to the sponge. This ensures an even coverage of the suture. The sutures are then air-dried to remove the water from the solution, thereby leaving rhGDF-5 adhered to the suture. The coated suture can then be used immediately for surgery.

In some embodiments, the sponge has a rounded outer surface 30 for ease of use.

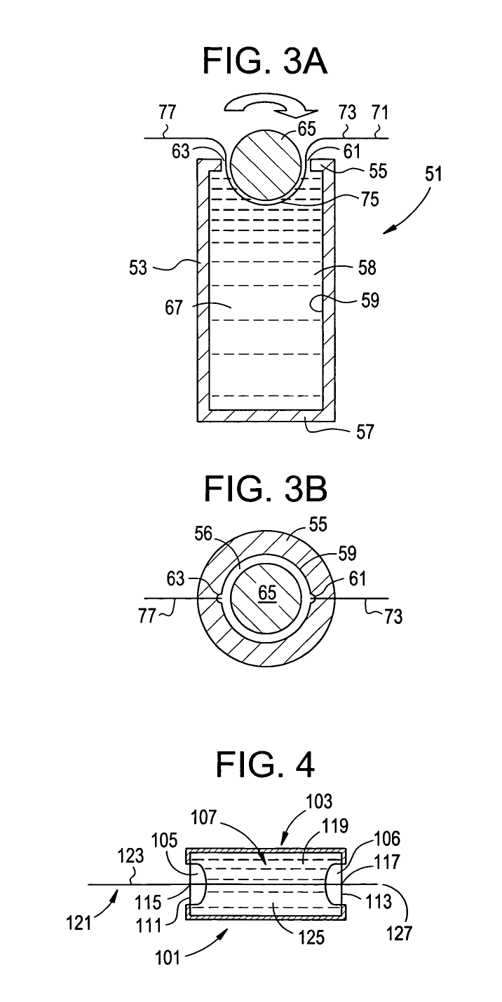

Now referring to FIGS. 3a and 3b, there is provided a device 51 for coating a suture, comprising:
a) a vessel 53 having an open end 55 having an opening 56, a closed end 57, and an inner wall 59 defining a chamber 58,
b) a roller ball 65 disposed within the opening;
c) an entry recess 61 and an exit recess 63 extending into the inner wall at the opening,
d) a fluid 67 comprising a therapeutic agent contained within the chamber, and
e) a suture 71 having i) a first end portion 73 extending out of the entry recess, ii) an intermediate portion 75 passing through the vessel and contacting the solution and iii) a second end portion 77 exiting the vessel through the exit recess.

In this third embodiment, rhGDF-5 is the therapeutic agent and is present in a reconstituted form in a vessel 53 with sterile saline. As the sutures are pulled through, they are evenly coated with rhGDF-5. The sutures are then air-dried to remove the water from the solution, thereby leaving rhGDF-5 adhered to the suture. The coated suture can then be used immediately for surgery.

Preferably, the inner wall of this device has a concave curve (not shown) at the open end of the vessel.

Preferably, the entry and exit recesses are substantially opposite one another at the open end of the vessel.

In some embodiments, the second end portion of the suture is contained on a roll (not shown). In some embodiments, the inner wall has a diameter at the open end that is slightly larger than the diameter of the roller ball.

Now referring to FIG. 4, there is provided a device 101 for coating a suture, comprising:
a) a vessel 103 having a longitudinal axis, a first open end 105 and a second open end 106, and a chamber 107 therebetween,
b) first 111 and second 113 septa respectively enclosing the first and second open ends, the first septum having a first hole 115 extending from the chamber to first open end and the second septum having a second hole 117 extending from the chamber to second open end,
c) a fluid 119 (such as a thermoreversible gel) containing a therapeutic agent (such as a growth factor) contained within the chamber of the vessel, and d) a suture 121 having i) a first end portion 123 extending out of the first open end, ii) an intermediate portion 125 passing through the chamber and contacting the solution and iii) a second end portion 127 exiting the vessel through the second open end.

In preferred fourth embodiments, the vessel is a vial that contains two septa at opposite ends. Contained in the vial is a sterile solution of rhGDF-5 and a thermo-reversible gel. The gelling point of the gel is above room temperature. At the time of intra-operative use, the vial is heated above the gelling point of the gel to liquify it. The suture is passed through the vial via the septum to insure an even coating. In some embodiments, the suture may be attached to a needle and the needle is passed through the septa before the suture. Once the liquefied solution emerges from the vial, the coating solidifies on the suture as its temperature proceeds to room temperature. The suture is then immediately used in surgery.

In one example, the thermoreversible gel is a medical grade gelatin. Preferably, the gelation point of the gelatin is about 30° C.

In some embodiments, the first and second holes are substantially opposite one another along the longitudinal axis.

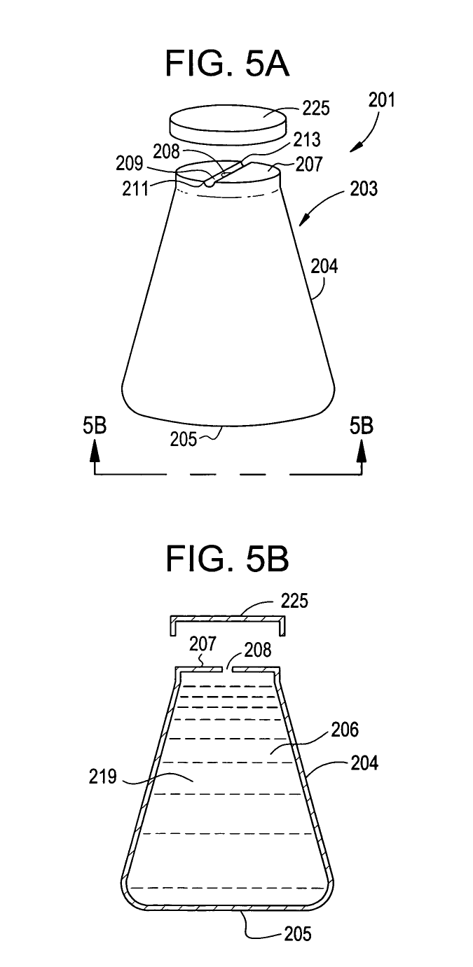

Now referring to FIGS. 5a and 5b, there is provided a device 201 for coating a suture, comprising:
a) a vessel 203 having a bottom surface, 205, a top surface 207, a flexible wall 204 therebetween defining first 211 and second 213 edges with the top surface and a chamber 206, a first opening 208 in the top surface in fluid communication with the chamber, and a linear recess 209 in the top surface that passes across the first opening and intersects the first and second edges, and
b) a fluid 219 (such as a thermoreversible gel) containing a therapeutic agent (such as a growth factor) contained within the chamber of the vessel, and
c) a cap 225 adapted to enclose the top surface of the vessel.

In use, the cap is removed from the top surface of the vessel to expose the first opening. A suture to be coated is placed along the linear recess in the top surface of the vessel. The flexible wall of the vessel is then squeezed to force fluid from the chamber to flow out of the first opening, thereby coating the suture in the vicinity of the opening. Other portions of the suture are then passed over the opening, guided by the linear recess, and are likewise coated.

In some preferred embodiments, the therapeutic agent to be coated upon the suture is a protein. In some embodiments, the therapeutic protein to be coated upon the suture is selected from the group consisting of growth factors, anti-microbials, analgesics, anti-inflammatory agents, anti-neoplastics, RGD sequences, fibrin and clotting factors.

In some embodiments, the therapeutic agent to be coated upon the suture is selected from the group consisting of amino acids, anabolics, analgesics and antagonists, anaesthetics, anti-adrenergic agents, anti-asthmatics, anti-atherosclerotics, antibacterials, anticholesterolics, anti-coagulants, antidepressants, antidotes, anti-emetics, anti-epileptic drugs, anti-fibrinolytics, anti-inflammatory agents, antihypertensives, antimetabolites, antimigraine agents, antimycotics, antinauseants, antineoplastics, anti-obesity agents, antiprotozoals, antipsychotics, antirheumatics, antiseptics, antivertigo agents, antivirals, appetite stimulants, bacterial vaccines, bioflavonoids, calcium channel blockers, capillary stabilizing agents, coagulants, corticosteroids, detoxifying agents for cytostatic treatment, diagnostic agents (like contrast media, radiopaque agents and radioisotopes), electrolytes, enzymes, enzyme inhibitors, ferments, ferment inhibitors, gangliosides and ganglioside derivatives, hemostatics, hormones, hormone antagonists, hypnotics, immunomodulators, immunostimulants, immunosuppressants, minerals, muscle relaxants, neuromodulators, neurotransmitters and nootropics, osmotic diuretics, parasympatholytics, para-sympathomimetics, peptides, proteins, psychostimulants, respiratory stimulants, sedatives, serum lipid reducing agents, smooth muscle relaxants, sympatholytics, sympathomimetics, vasodilators, vasoprotectives, vectors for gene therapy, viral vaccines, viruses, vitamins, oligonucleotides and derivatives, and any therapeutic agent capable of affecting the nervous system.

In some embodiments, the therapeutic agent to be coated upon the suture is a non-curing therapeutic agent.

Any biocompatible fluid capable of coating a suture may be used in accordance with the present invention. Suitable fluids include aqueous liquids (such as saline) and gels that include, but are not limited to, hyaluronic acid, succinalyted collagen, carboxymethyl cellulose (CMC), gelatin, collagen gel, fibrinogen/thrombin, and liquid polymers (MGSA).

Porous bodies suitable for use in the present invention include but are not limited to sponges, membranes, pads, non-woven materials, felt, meshes, knit meshes, woven materials and braided materials.

Preferably, the sutures used in accordance with the present invention will be bioresorbable. However, the sutures may also be non-resorbable. Preferred bioresorbable materials which can be used to make the sutures of the present invention include bioresorbable polymers or copolymers, preferably selected from the group consisting of hydroxy acids, (particularly lactic acids and glycolic acids; caprolactone; hydroxybutyrate; dioxanone; orthoesters; orthocarbonates; and aminocarbonates). Preferred bioresorbable materials also include natural materials such as chitosan, collagen, cellulose, fibrin, hyaluronic acid; fibronectin, and mixtures thereof. However, synthetic bioresorbable materials are preferred because they can be manufactured under process specifications which insure repeatable properties. (see http://www.emedicine.com/derm/topic825.htm for listing of nonresorbable materials)

A variety of bioabsorbable polymers can be used to make the suture of the present invention. Examples of suitable biocompatible, bioabsorbable polymers include but are not limited to polymers selected from the group consisting of aliphatic polyesters, poly(amino acids), copoly(ether-esters), polyalkylenes oxalates, polyamides, tyrosine derived polycarbonates, poly(iminocarbonates), polyorthoesters, polyoxaesters, polyamidoesters, polyoxaesters containing amine groups, poly(anhydrides), polyphosphazenes, biomolecules (i.e., biopolymers such as collagen, elastin, bioabsorbable starches, etc.) and blends thereof. For the purpose of this invention aliphatic polyesters include, but are not limited to, homopolymers and copolymers of lactide (which includes lactic acid, D-,L- and meso lactide), glycolide (including glycolic acid), ε-caprolactone, p-dioxanone (1,4-dioxan-2-one), trimethylene carbonate (1,3-dioxan-2-one), alkyl derivatives of trimethylene carbonate, δ-valerolactone, β-butyrolactone, χ-butyrolactone, ε-decalactone, hydroxybutyrate, hydroxyvalerate, 1,4-dioxepan-2-one (including its dimer 1,5,8,12-tetraoxacyclotetradecane-7,14-dione), 1,5-dioxepan-2-one, 6,6-dimethyl-1,4-dioxan-2-one, 2,5-diketomorpholine, pivalolactone, χ,χ-diethylpropiolactone, ethylene carbonate, ethylene oxalate, 3-methyl-1,4-dioxane-2,5-dione, 3,3-diethyl-1,4-dioxan-2,5-dione, 6,8-dioxabicycloctane-7-one and polymer blends thereof. Poly(iminocarbonates), for the purpose of this invention, are understood to include those polymers as described by Kemnitzer and Kohn, in the Handbook of Biodegradable Polymers, edited by Domb, et. al., Hardwood Academic Press, pp. 251-272 (1997). Copoly(ether-esters), for the purpose of this invention, are understood to include those copolyester-ethers as described in the Journal of Biomaterials Research, Vol. 22, pages 993-1009, 1988 by Cohn and Younes, and in Polymer Preprints (ACS Division of Polymer Chemistry), Vol. 30(1), page 498, 1989 by Cohn (e.g. PEO/PLA). Polyalkylene oxalates, for the purpose of this invention, include those described in U.S. Pat. Nos. 4,208,511; 4,141,087; 4,130,639; 4,140,678; 4,105,034; and 4,205,399. Polyphosphazenes, co-, ter- and higher order mixed monomer-based polymers made from L-lactide, D,L-lactide, lactic acid, glycolide, glycolic acid, para-dioxanone, trimethylene carbonate and α-caprolactone such as are described by Allcock in The Encyclopedia of Polymer Science, Vol. 13, pages 31-41, Wiley Intersciences, John Wiley & Sons, 1988 and by Vandorpe, et al in the Handbook of Biodegradable Polymers, edited by Domb, et al, Hardwood Academic Press, pp. 161-182 (1997). Polyanhydrides include those derived from diacids of the form HOOC—C₆H₄—O—(CH₂)ₘ—O—C₆H₄—COOH, where m is an integer in the range of from 2 to 8, and copolymers thereof with aliphatic alpha-omega diacids of up to 12 carbons. Polyoxaesters, polyoxaamides and polyoxaesters containing amines and/or amido groups are described in one or more of the following U.S. Pat. Nos. 5,464,929; 5,595,751; 5,597,579; 5,607,687; 5,618,552; 5,620,698; 5,645,850; 5,648,088; 5,698,213; 5,700,583; and 5,859,150. Polyorthoesters such as those described by Heller in Handbook of Biodegradable Polymers, edited by Domb, et al, Hardwood Academic Press, pp. 99-118 (1997).

Preferably, the bioresorbable material is selected from the group consisting of poly(lactic acid) (“PLA”) and poly(glycolic acid)(“PGA”), and copolymers thereof.

In some preferred embodiments, the suture comprises collagen because rhGDF-5 has a high affinity towards collagen. In some preferred embodiments, the suture comprises surgical gut, which comprises purified connective tissue (of which its main component is type I collagen) derived from either the serosal layer or the submucosal fibrous layer of bovine intestines.

Therefore, in accordance with the present invention, there is provided a suture comprising collagen coated with rhGDF-5.

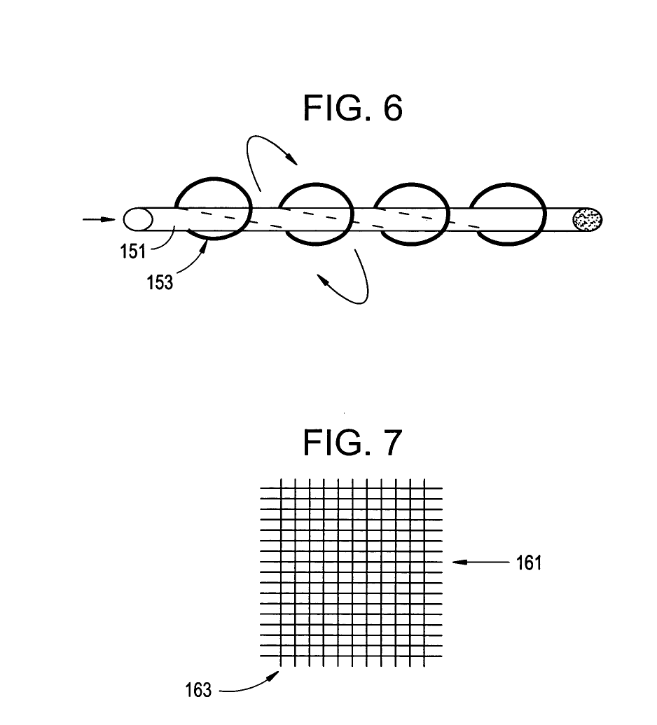

In some embodiments and now referring to FIG. 6, regular sterile sutures 151 are wrapped or interwoven with sterile surgical gut suture 153, and then this composite construct is coated with rhGDF-5 as per the first embodiment described above.

In other embodiments, and now referring to FIG. 7, strands of surgical gut sutures 161 can be manufactured and/or interwoven with other synthetic fibers 163 to create a mesh or non-woven composite structure. This mesh/non-woven composite would have enhanced binding collagen capacity on the surface for rhGDF-5 coating. Then, this mesh/non-woven composite construct is coated with rhGDF-5 as per the second embodiment described above.

Therefore, in accordance with the present invention, there is provided a composite suture comprising a first suture comprising collagen (which is preferably surgical gut) and a second suture, wherein the composite suture is coated with rhGDF-5.

When gut suture is selected, in some embodiments, it is Plain Gut Suture-Surgical Gut Suture Absorbable, USP, available from Ethicon (Somerville, N.J.). Plain gut suture is a strand of collagenous material prepared from the submucosal layers of the small intestine of healthy sheep, or from the serosal layers of the small intestine of healthy cattle. They are sterile and elicit only a slight to minimal tissue reaction during absorption. Fast absorbing surgical gut sutures differ from U.S.P. minimum strength requirements by less than 30%.

When gut suture is selected, in some embodiments, it is Chromic Gut Suture-Surgical Gut Suture Absorbable, USP, available from Ethicon (Somerville, N.J.). Chromic gut suture is an absorbable, sterile surgical suture composed of purified connective tissue (mostly collagen) derived from either the serosal layer of beef (bovine) or the submucosal fibrous layer of sheep (ovine) intestines. Surgical gut sutures are available in plain or chromic. Chromic gut is processed to provide greater resistance to absorption. Surgical gut is packaged in tubing fluid. Blue dyed chromic gut suture is also available. Surgical gut suture meets all requirements established by the U.S. Pharmacopeia (U.S.P.) for absorbable surgical sutures.

As used herein, the term “growth factors” encompasses any cellular product that modulates the adhesion, migration, proliferation, or differentiation of other cells, particularly connective tissue progenitor cells. The growth factors that may be used in accordance with the present invention include, but are not limited to, members of the fibroblast growth factor family, including acidic and basic fibroblast growth factor (FGF-1 and -2) and FGF-4, members of the platelet-derived growth factor (PDGF) family, including PDGF-AB, PDGF-BB and PDGF-AA; EGFs, members of the insulin-like growth factor (IGF) family, including IGF-I and -II; the TGF-β superfamily, including TGF-β1, 2 and 3 (including rhGDF-5), osteoid-inducing factor (OIF), angiogenin(s), endothelins, hepatocyte growth factor and keratinocyte growth factor; members of the bone morphogenetic proteins (BMP's) BMP-1, (BMP-3); BMP-2; OP-1; BMP-2A, -2B, and -7, BMP-14; HBGF-1 and -2; growth differentiation factors (GDF's), members of the hedgehog family of proteins, including indian, sonic and desert hedgehog; ADMP-1; members of the interleukin (IL) family, including IL-1 thru -6; members of the colony-stimulating factor (CSF) family, including CSF-1, G-CSF, GM-CSF, VEGF integrin binding sequence, ligands, bone morphogenic proteins, epidermal growth factor, IGF-I, IGF-II, TGF-β I-III, growth differentiation factor, parathyroid hormone, hyaluronic acid, glycoprotein, lipoprotein, OP-1, BMP-2, BMP-4, BMP-6, BMP-12, GDF5, GDF6, GDF8, PDGF, small molecules that affect the upregulation of specific growth factors, tenascin-C, fibronectin, thromboelastin, thrombin-derived peptides, heparin-binding domains, and isoforms thereof.)

In some embodiments, the growth factor is GDF-5, preferably rhGDF-5. More preferably, the rhGDF-5 is administered using a solution with concentrations between 10 ng/mL and 40 mg/mL, more preferably between 100 ng/mL and 5 mg/mL, more preferably between 1 μg/mL and 1 mg/mL.

In some instances, the surgeon may want to coat just a portion of the suture. In others, the surgeon may want to insure that the suture has been evenly coated with the growth factor. In others, the surgeon may want to know simply when the suture has been sufficiently coated so that additional application of an expensive therapeutic agent is not required. These desires can be realized if the surgeon were able to see the extent to which the suture has been coated. Accordingly, in some embodiments, a dye may be added to the coating solution so that the surgeon can visualize the extent to which the suture has been coated. Suitable dyes include but are not limited to isosulfan blue (which has been used to visualize lymph nodes in breast cancer patients) and India ink.

Although the foregoing discussion has focused upon the coating of sutures, it should be appreciated by one skilled in the art that any number of medical devices or implants may benefit by being coated with the coatings of this invention incorporated therewith to enhance the function and/or life of the device or implant. Other medical devices which often fail due to tissue in-growth or accumulation of proteinaceous material in, on and around the device, such as stents, dialysis grafts, colostomy bag attachment devices, ear drainage tubes, leads for pace makers and implantable defibrillators can also benefit from the coatings of this invention. Devices which serve to improve the structure and function of tissue or organ may also show benefits when combined with the appropriate agent or agents. For example, improved osteointegration of orthopedic devices to enhance stabilization of the implanted device could potentially be achieved by combining it with agents such as bone-morphogenic protein. Similarly other surgical devices, sutures, staples, fasteners, vertebral motion discs, vertebral fusion cages and meshes, hooks, rods, pedicle screws, bone pins, suture anchors, hemostatic barriers, clamps, screws, plates, clips, vascular implants, tissue adhesives and sealants, tissue scaffolds, various types of dressings, bone substitutes, intraluminal devices, and vascular supports could also provide enhanced patient benefit using coatings of this invention. Essentially, any type of medical device may be coated in some fashion with coatings of this invention which enhance treatment over use of the singular use of the device or implant.

### EXAMPLE I

A 4-0 VICRYL (Polyglactin 910) Suture (Ethicon, Somerville, N.J.) was coated with rhGDF-5 and gelatin. The coating solution comprised of 4 ml gelatin solution and 2 ml of rhGDF-5 growth factor solution. The gelatin component was prepared by heating a 10 wt % solution of medical grade soluble bovine collagen (Semed-S, Kensey-Nash, Exton, Pa.) to 80° C. for 10 minutes followed by incubation at 37° C. rhGDF-5 (Biopharm GmbH, Heidelberg, Germany) was reconstituted with 10 mM HCl at concentrations of 3, 0.6, and 0 mg/ml. The resulting concentrations in the coating solutions were 1000, 200, and 0 μg/ml, respectively. The coating solutions were kept at 37° C. until use.

Prior to coating, the sutures were pretreated with a bath of 70% ethanol solution for 10 minutes, followed by a wash with saline. The suture was then placed in the coating solution and incubated at 37° C. for 30 minutes with gentle agitation. The suture was then removed from the solution and was then air-dried overnight.

The concentration of rhGDF-5 on the suture was quantified by an ELISA method. The growth factor was first eluted from a 4 cm segment of suture in 2 ml of 6M Urea solution (75 mM NaH₂PO₄, pH 2.7) at 37° C. for 1 hour. The elution solutions were analyzed by sandwich ELISA (Biopharm GmbH, Heidelberg, Germany) that detects rhGDF-5. The concentration of rhGDF-5 on the suture was presented in μg/cm. The results are presented in Table I.

TABLE 1
ELISA quantification of rhGDF-5 on
gelatin/rhGDF-5 coated suture.

| | rhGDF-5 Concentration on |
|---|---|
| Coating Solution (μg/ml) | Suture (μg/cm) |
| 0 | −0.020 |
| 200 | 0.030 |
| 1000 | 0.522 |

### EXAMPLE II

A 0 ETHIBOND EXCEL Polyester Suture (Ethicon, Somerville, N.J.) was coated with rhGDF-5 and gelatin in a similar manner as described in Example I. A rhGDF-5 solution was concentrated to 30 mg/ml with a centrifugal filter device (Centriplus YM-10, Regenerated Cellulose 10,000 MWCO, Amicon Bioseparations). The coating solution comprised of 0.5 ml concentrated rhGDF-5 solution and 1 ml 10 wt % gelatin solution. The concentration of rhGDF-5 on the coated suture, as quantified by ELISA, was 6.5 μg/cm.

Sutures were pulled through goat ACL tissue to evaluate if any of the growth factor coating is sheared off during its use. The concentration of rhGDF-5 post-surgery was 5.9 μg/cm, indicating that gelatin is effective in maintaining the growth factor on the suture even while passing through tissue.

### EXAMPLE III

A 0 Plain Surigcal Gut Suture (Ethicon, Somerville, N.J.) was coated with rhGDF-5. The coating solution comprised of 1 ml rhGDF-5 solution concentrated to 13.9 mg/ml with a centrifugal filter device (Centriplus YM-10, Regenerated Cellulose 10,000 MWCO, Amicon Bioseparations). The gut suture was pretreated in a bath of 200 mM NaH₂PO₄ (pH 11.2) for 10 minutes followed by a wash in PBS prior to coating. The concentration of rhGDF-5 on the coated gut suture, as quantified by ELISA, was 26.3 μg/cm.

We claim:

1. A device for coating a suture, comprising:
a) a vessel having an open end,
b) a porous body enclosing the open end of the vessel; and
c) a therapeutic protein contained within the vessel.

2. The device of claim 1 wherein the vessel is a vial.

3. The device of claim 1 wherein the porous body is a sponge.

4. The device of claim 3 wherein the sponge has an outer surface having a recess.

5. The device of claim 1 wherein a dye is contained within the vessel.

6. The device of claim 1 wherein the therapeutic protein is a growth factor.

7. A method of coating a suture, comprising the steps of:
a) providing a device comprising i) a vessel having an open end, ii) a porous body enclosing the open end of the vessel; and iii) a therapeutic agent contained within the vessel,
b) injecting a fluid into the vessel to produce a solution comprising reconstituted solution,
c) contacting the solution with the porous body to wet the porous body,
d) contacting the suture with thewetted porous body.

8. The method of claim 7 wherein the fluid is an aqueous fluid.

9. The method of claim 7 wherein the therapeutic agent is a growth factor.

10. The method of claim 7 wherein the therapeutic agent is butyric acid.

11. A device for coating a suture, comprising:
a) a vessel having an open end and an inner wall,
b) a porous body enclosing the open end of the vessel;
c) a breakable seal disposed upon the inner wall of the vessel defining a first chamber and a second chamber,
d) a fluid contained within the first chamber, and
e) a therapeutic protein contained within the second chamber.

12. The device of claim 11 wherein the vessel is a vial.

13. The device of claim 11 wherein the porous body is a sponge.

14. The device of claim 13 wherein the sponge has a rounded outer surface.

15. The device of claim 11 wherein the fluid includes a dye.

16. The device of claim 11 wherein the therapeutic protein is a growth factor.

17. The device of claim 11 wherein the aqueous fluid is saline.

18. A method of coating a suture, comprising the steps of:
a) providing a device comprising:
i) a vessel having an open end and an inner wall,
ii) a porous body enclosing the open end of the vessel;
iii) a breakable seal disposed upon the inner wall of the vessel defining a first chamber and a second chamber,
iv) a fluid contained within the first chamber, and
v) a therapeutic agent contained within the second chamber,
b) breaking the seal between the chamber to mix the fluid and the therapeutic agent and wet the porous body.
c) contacting the suture with the wetted porous body.

19. The method of claim 18 wherein the therapeutic agent is a therapeutic protein.

20. The method of claim 18 wherein the therapeutic protein is a growth factor.

21. A device for coating a suture, comprising:
a) a vessel having an open end having an opening, a closed end, and an inner wall defining a chamber,
b) a roller ball disposed within the opening and having a diameter;
c) an entry recess and an exit recess extending into the inner wall at the opening,
d) a fluid comprising a therapeutic agent contained within the chamber, and
e) a suture having i) a first end portion extending out of the entry recess, ii) an intermediate portion passing through the vessel and contacting the solution and iii) a second end portion exiting the vessel through the exit recess.

22. The device of claim 21 wherein the vessel is a vial.

23. The device of claim 21 wherein the inner wall has a concave curve at the open end of the vessel.

24. The device of claim 23 wherein the entry and exit recesses are substantially opposite one another at the open end of the vessel.

25. The device of claim 21 wherein the therapeutic agent is a growth factor.

26. The device of claim 21 wherein the fluid is saline.

27. The device of claim 21 wherein the fluid contains a dye.

28. The device of claim 27 wherein the second end portion of the suture is contained on a roll.

29. The device of claim 21 the inner wall has a diameter at the open end that is slightly less than the diameter of the roller ball.

30. A method of coating a suture, comprising the steps of:
a) providing the device of claim 21,
b) passing the intermediate portion of the suture through the exit recess.

31. A device for coating a suture, comprising:
a) a vessel having a first open end and a second open end, and a chamber therebetween,
b) a fluid comprising a therapeutic agent contained within the chamber of the vessel, and
c) a suture having i) a first end portion extending out of the first open end, ii) an intermediate portion passing through the chamber and contacting the fluid and iii) a second end portion exiting the vessel through the second open end.

32. The device of claim 31 wherein the fluid comprises a thermoreversible gel.

33. The device of claim 31 wherein the first and second open ends are respectively enclosed by first and second septa, the first septum having a first hole extending from the chamber to first open end and the second septum having a second hole extending from the chamber to second open end.

34. The device of claim 31 wherein the vessel has a longitudinal axis, and the first and second holes are substantially opposite one another along the longitudinal axis.

35. The device of claim 31 wherein the therapeutic agent is a growth factor.

36. A method of coating a suture, comprising the steps of:
a) providing the device of claim 32,
b) heating the thermoreversible gel above its gelling point, thereby coating the intermediate portion of the suture, and
c) pulling the second end portion of the suture in order to pass the intermediate portion of the suture coated with the solution through the second open end.

37. A composite comprising a first suture comprising collagen and a second suture, wherein the composite is coated with rhGDF-5.

38. The composite of claim 37 wherein the first suture is surgical gut.

39. The composite of claim 38 wherein the first and second sutures are woven together.

40. The composite of claim 38 wherein the first and second sutures form a non-woven mesh structure.

41. A method comprising intra-operatively coating a suture with a fluid comprising a growth factor.

42. A suture comprising a first portion coated with a growth factor and a second portion uncoated by the growth factor.

43. A method of coating a suture, comprising the steps of:
a) providing a device comprising i) a vessel having an opening, and ii) a therapeutic agent contained within the vessel and in fluid communication with the opening,
b) contacting the suture to the opening of the vessel.

44. The method of claim 43 wherein the therapeutic agent is a growth factor.

45. The method of claim 43 wherein the therapeutic agent is within a TGF-β superfamily.

46. The method of claim 43 wherein the therapeutic agent is a BMP.

47. The method of claim 43 wherein the therapeutic agent is a BMP-2.

48. The method of claim 43 wherein the therapeutic agent is a BMP-7.

49. The method of claim 43 wherein the therapeutic agent is butyric acid.

50. The method of claim 43 wherein the therapeutic agent is GDF-5.

51. The method of claim 43 wherein the therapeutic agent is OP-1.

52. The method of claim 43 wherein the therapeutic agent is VEGF.

53. The method of claim 43 wherein the therapeutic agent is PDGF.

54. A device for coating a suture, comprising:
a) a vessel having a flexible wall defining a chamber, an end surface, and a first opening in the end surface in fluid communication with the chamber,
b) a fluid containing a therapeutic protein contained within the chamber of the vessel.

55. The device of claim 54 further comprising:
c) a cap adapted to enclose the end surface of the vessel.

56. The device of claim 54 wherein the end surface comprises a linear recess.

57. The device of claim 54 wherein the fluid comprises a thermoreversible gel.

58. The device of claim 54 wherein the therapeutic protein is a growth factor.

59. A method of coating a suture, comprising the steps of:
a) providing a device containing a therapeutic agent, and
b) contacting the suture to the device.
